(19) United States
(12) Patent Application Publication
(10) Pub. No.: US 2001/0024655 A1
(43) Pub. Date: Sep. 27, 2001

(54) Aqueous-alcoholic preparations containing chitosan

(76) Inventors: Gunther Schneider, Hamburg (DE); Rainer Kropke, Schenefeld (DE); Waltraud Kaden, Halstenbek (DE); Gabriele Winkler, Wedel (DE)

Correspondence Address: Norris McLaughlin & Marcus, P.A., 660 White Plains Road, Tarrytown, NY, 10591-5144, US

(21) Appl. No.: 09/797,492

(22) Filed: Mar. 1, 2001

(30) Foreign Application Priority Data
Mar. 2, 2000 (DE) 100 10 199.2

(51) Int. Cl. A01N 43/04; A61K 31/74; A61K 6/00; A61K 7/00

(52) U.S. Cl. 424/401; 424/78.03; 514/55

(57) ABSTRACT

Cosmetic face tonics comprising

a) chitosan having an average molecular weight of from 10 000 to 1 000 000 g/mol and a degree of deacylation of from 10 to 99%

b) 10-80% by weight of ethanol

c) 0-5% by weight of one or more lipids,

d) 0-5% by weight of one or more fatty alcohols.

The present invention relates to cosmetic and dermatological preparations containing chitosan.

The skin is the largest human organ. Of its many functions (for example for heat regulation and as a sensory organ), the barrier function, which prevents the skin (and thus ultimately the entire organism) from drying out, is probably the most important. At the same time the skin acts as a protective device against the penetration and the absorption of external substances. This barrier function is effected by the epidermis which, being the outermost layer, forms the actual protective sheath against the environment. Being about one tenth of the overall thickness, it is also the thinnest layer of the skin.

The epidermis is a stratified tissue in which the outer layer, the horny layer (Stratum corneum), represents the part which is of significance for the barrier function. The Elias skin model, which is currently recognized in the specialist field (P. M. Elias, Structure and Function of the Stratum Corneum Permeability Barrier, Drug Dev. Res. 13, 1988, 97-105), describes the horny layer as a two-component system, similar to a brick wall (bricks and mortar model). In this model the horny cells (corneocytes) correspond to the bricks, and the lipid membrane, which is of complex composition, in the intercellular spaces corresponds to the mortar. This system is essentially a physical barrier against hydrophilic substances but, because of its narrow and multilayered structure, can equally, however, also only be passed by lipophilic substances with difficulty.

The present invention relates, in a particular embodiment, to cosmetic or pharmaceutical preparations having a reduced feel of stickiness, to processes for their preparation, and to the use of active ingredients for reducing the feel of stickiness of cosmetic preparations.

Cosmetic skincare is primarily understood as meaning that the natural function of the skin has a barrier against environmental influences (e.g. dirt, chemicals, microorganisms) and against the loss of endogenous substances (e.g. water, natural fats, electrolytes) is strengthened or restored.

If this function is impaired, increased resorption of toxic or allergenic substances or attack by microorganisms may result, leading to toxic or allergic skin reactions.

Another aim of skincare is to compensate for the loss by the skin of fats and water caused by daily washing. This is particularly important if the natural regeneration ability is inadequate. Furthermore, skincare products should protect against environmental influences, in particular against sun and wind, and delay skin aging.

Aqueous alcoholic solutions form the basis of tonics. They are mostly applied as the final step of face care in order to impart a feeling of freshness and a refreshed appearance to the face. In addition to perfume and other additives, tonics generally comprise humectants, such as, for example, glycerol.

The alcohol content of face tonics is usually in the range 10-20% by weight. A higher content of ethanol would sometimes be desirable in order to enable certain formulation constituents, for example perfume constituents, to be more readily soluble, an increased ethanol content on the other hand not infrequently leads to skin irritation, at least in the case of sensitive skin.

The use of chitosan in cosmetic preparations is known per se. Chitosan is a partially deacylated chitin. This biopolymer has, inter alia, film-forming properties and is characterized by a silky feel on the skin. however, a disadvantage is its considerable stickiness on the skin which occurs in particular—temporarily—during use. In isolated instances it is not possible to market corresponding preparations since they are not accepted or viewed negatively by the consumer. As is known, chitosan is used, for example, in haircare. It is suitable, more so than the chitin on which it is based, as a thickener or stabilizer and improves the adhesion and water resistance of polymeric films. A representative of a large number of literature references for the prior art is: H. P. Fiedler, “Lexikon der Hilfsstoffe fur Pharmazie, Kosmetik und angrenzende Gebiete” [Lexikon of Auxiliaries for Pharmacy, Cosmetic and Related Fields], 3rd Edition 1989, Editio Cantor, Aulendorf, p. 293, keyword “Chitosan”.

Chitosan is characterized by the following structural formula:

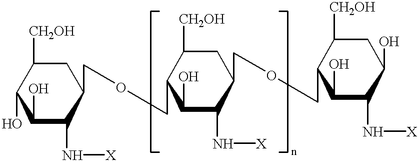

where n assumes values up to about 10 000, X is either the acetyl radical or hydrogen. Chitosan is formed by the deacetylation and partial depolymerization (hydrolysis) of chitin, which is characterized by the structural formula

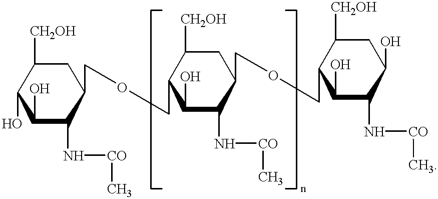

Chitin is an essential constituent of the ectoskeleton ['οχιτων=Greek: integument] of arthropods (e.g. insects, crabs, spiders) and is also found in supporting tissues of other organisms (e.g. mollusks, algae, fungi).

In the region of about pH <6, chitosan is positively charged and in that range is also soluble in aqueous systems. It is incompatible with anionic raw materials. Therefore, for the preparation of chitosan-containing oil-in-water emulsions, the use of nonionic emulsifiers is appropriate. Such emulsifiers are known per se, for example from EP-A 776 657.

The emulsions listed therein (e.g. with the O/W emulsifier cetylstearylglucoside in a mixture with cetylstearyl alcohol) do, however, have certain disadvantages with regard to their sensory quality.

The object of the invention was therefore to remedy this shortcoming and to provide face tonics or alcoholic tonics which on the one hand, when used in accordance with the directions, impart a feeling of freshness and a refreshed appearance to the face, but without having to buy formulation-related advantages at the cost of an unpleasant feel on the skin, for example stickiness, or even reduced skin friendliness.

Surprisingly, these objects are achieved by cosmetic face tonics comprising

a) chitosan having an average molecular weight of from 10 000 to 1 000 000 g/mol and a degree of deacylation of from 10 to 99%

b) 10-80% by weight of ethanol

c) 0-5% by weight of one or more lipids,

d) 0-5% by weight of one or more fatty alcohols.

It was surprising that the preparations according to the invention do not have a skin-irritative action even when the alcohol contents are relatively high and that, even at relatively high chitosan contents, no sticky feel on the skin arises. Instead, according to the invention, face tonics are obtainable which bring about a silky feel on the skin and impart a feeling of freshness and a fresh appearance to the skin of the face.

The fatty alcohol or alcohols whose use is in itself optional is or are preferably chosen from the group of branched and unbranched alkyl alcohols having 12 to 40 carbon atoms.

A preferred fatty alcohol used according to the invention is cetylstearyl alcohol (a mixture of 1-hexadecanol and 1-octadecanol in equal parts).

The total amount of one or more fatty alcohols used according to the invention in the finished cosmetic or dermatological preparations is advantageously chosen from the range 0.1-5.0% by weight, preferably 0.5-1.0% by weight, based on the total weight of the preparations.

The lipid component or components whose use in itself is optional are preferably chosen from the group of customary cosmetic oil components.

The lipid phase can advantageously be chosen from the following group of substances:

mineral oils, mineral waxes

oils, such as triglycerides of capric or of caprylic acid, but preferably castor oil;

fats, waxes and other natural and synthetic fatty substances, preferably esters of fatty acids with alcohols of low carbon number, e.g. with isopropanol, propylene glycol or glycerol, or esters of fatty alcohols with alkanoic acids of low carbon number or with fatty acids;

alkyl benzoates;

silicone oils, such as dimethylpolysiloxanes, diethylpolysiloxanes, diphenylpolysiloxanes and mixed forms thereof.

The oil components can also advantageously be chosen from the group of esters of saturated and/or unsaturated, branched and/or unbranched alkanecarboxylic acids having a chain length of from 3 to 30 carbon atoms and saturated and/or unsaturated, branched and/or unbranched alcohols having a chain length of from 3 to 30 carbon atoms, from the group of esters from the aromatic carboxylic acids and saturated and/or unsaturated, branched and/or unbranched alcohols having a chain length of from 3 to 30 carbon atoms. Such ester oils can then advantageously be selected from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, and synthetic, semisynthetic and natural mixtures of such esters, e.g. jojoba oil.

In addition, the oil components can advantageously be chosen from the group of branched and unbranched hydrocarbons and hydrocarbon waxes, silicone oils, dialkyl ethers, the group of saturated or unsaturated, branched or unbranched alcohols, and fatty acid triglycerides, namely the triglycerol esters of saturated and/or unsaturated, branched and/or unbranched alkanecarboxylic acids having a chain length of from 8 to 24, in particular 12-18, carbon atoms. The fatty acid triglycerides can, for example, be advantageously chosen from the group of synthetic, semisynthetic and natural oils, e.g. olive oil, sunflower oil, soybean oil, groundnut oil, rapeseed oil, almond oil, palm oil, coconut oil, palm kernel oil and the like.

For the purposes of the present invention, any mixtures of such oil and wax components can also be used advantageously. It may also, in some circumstances, be advantageous to use waxes, for example cetyl palmitate, as the sole lipid component of the oil phase.

The oil components are advantageously chosen from the group consisting of 2-ethylhexyl isostearate, octyldodecanol, isotridecyl isononanoate, isoicosane, 2-ethylhexyl cocoate, C₁₂₋₁₅-alkyl benzoate, caprylic/capric triglyceride, dicaprylyl ether.

Mixtures of C₁₂₋₁₅-alkyl benzoate and 2-ethylhexyl isostearate, mixtures of C₁₂₋₁₅-alkyl benzoate and isotridecyl isononanoate, and mixtures of C₁₂₋₁₅-alkyl benzoate, 2-ethylhexyl isostearate and isotridecyl isononanoate are particularly advantageous.

Of the hydrocarbons, paraffin oil, squalane and squalene are advantageously to be used for the purposes of the present invention.

The oil components may also advantageously have a content of cyclic or linear silicone oils or consist entirely of such oils, although it is preferable to use an additional content of other oil phase components apart from the silicone oil or the silicone oils.

Cyclomethicone (decamethylcyclopentasiloxane) is advantageously used as the silicone oil to be used according to the invention. However, other silicone oils can advantageously be used for the purposes of the present invention, for example hexamethylcyclotrisiloxane, more suitably octamethylcyclotetrasiloxane, polymethylsiloxane, poly(methylphenylsiloxane).

Mixtures of cyclomethicone and isotridecyl isononanoate, and mixtures of cyclomethicone and 2-ethylhexyl isostearate are also particularly advantageous.

Preference is given according to the invention to chitosans having a degree of deacetylation of >25%, in particular >55 to 99% [determined by means of ¹H-NMR].

It is advantageous to choose chitosans having molecular weights between 10 000 and 1 000 000, in particular those having molecular weights between 100 000 and 1 000 000 [determined by means of gel permeation chromatography].

According to the invention, cosmetic face tonics comprise 0.01 to 10% by weight, advantageously 0.1 to 5% by weight, very particularly preferably 0.25 to 2.5% by weight, of chitosans.

The chitosan is usually incorporated by adjusting chitosan in water to a pH of about 3.5-5.5 using organic or inorganic acids, giving, usually as a result of stirring, a solution of the chitosan. The clear solutions obtainable in this way are characterized, for example as a 2% strength by weight solution of chitosan in 1.2% lactic acid (90% strength aqueous solution), by a viscosity, according to a Viskotester-VT02 (Haake), which is in the range from 100 to 10 000 mPas, preferably from 200 to 5000 mPas.

It may, for example, be advantageous to use lactic acid, although it is likewise advantageous to use other acids which produce soluble chitosan salts, such as, for example, phosphoric acid, acetic acid, ascorbic acid (the latter preferably with use of a protective gas), hydrochloric acid, glycolic acid, nitric acid, 2-pyrrolidone-5-carboxylic acid, malic acid, salicylic acid, benzoic acid.

The person skilled in the art is of course aware that demanding cosmetic compositions are in most cases inconceivable without customary auxiliaries and additives. These include, for example, bodying agents, fillers, perfume, dyes, emulsifiers, additional active ingredients such as vitamins or proteins, light protection agents, stabilizers, insect repellants, alcohol, water, salts, antimicrobially, proteolytically or keratolytically active substances etc.

Accordingly, for the purposes of the present invention, cosmetic or topical dermatological compositions can, depending on their formulation, be used, for example, as skin protection cream, cleansing milk, sunscreen lotion, nourishing cream, day or night cream, etc. Where appropriate, it is possible and advantageous to use the compositions according to the invention as bases for pharmaceutical formulations.

The cosmetic and dermatological preparations according to the invention can comprise cosmetic active ingredients, auxiliaries and/or additives as are customarily used in such preparations, e.g. antioxidants, preservatives, bacteriocides, perfumes, antifoams, dyes, pigments which have a coloring action, thickeners, emollients, moisturizers and/or humectants, or other customary constituents of a cosmetic or dermatological formulation, such as alcohols, polyols, polymers, foam stabilizers, electrolytes, organic solvents or silicone derivatives.

For the purposes of the present invention, it is advantageous to add further anti-irritative or antiinflammatory active ingredients to the preparations, in particular batyl alcohol (α-octadecyl glyceryl ether), selachyl alcohol (α-9-octadecenyl glyceryl ether), chimyl alcohol (α-hexadecyl glyceryl ether), bisabolol and/or panthenol.

It is likewise advantageous to add customary antioxidants to the preparations for the purposes of the present invention. According to the invention, favorable antioxidants which may be used are all antioxidants customary or suitable for cosmetic and/or dermatological applications.

The antioxidants are advantageously chosen from the group consisting of amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoids, carotenes (e.g. α-carotene, β-carotene, Ψ-lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof, and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (e.g. buthionine sulfoximines, homocysteine sulfoximine, buthionine sulfones, penta-, hexa-, heptathionine sulfoximine) in very low tolerated doses (e.g. pmol to μmol/kg), and also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, furfurylidenesorbitol and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate), and coniferyl benzoate of benzoin resin, rutic acid and derivatives thereof, α-glycosylrutin, ferulic acid, furfurylideneglucitol, carnosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide) and the derivatives suitable according to the invention (salts, esters, ethers, sugars, nucleotides, nucleosides, peptides and lipids) of said active ingredients.

The amount of antioxidants (one or more compounds) in the preparations is preferably 0.001 to 30% by weight, particularly preferably 0.05-20% by weight, in particular 1-10% by weight, based on the total weight of the preparation.

If vitamin E and/or derivatives thereof are the antioxidant or antioxidants, it is advantageous to choose their respective concentrations from the range 0.001-10% by weight, based on the total weight of the formulation.

For the purposes of the present invention, the water phase of the cosmetic preparations can also have a gel character which, in addition to an effective content of substances to be used according to the invention and solvents customarily used therefor, preferably water, also comprises further organic thickeners, e.g. gum arabic, xanthan gum, sodium alginate, starch and starch derivatives (e.g. distarch phosphate), cellulose, cellulose derivatives, preferably methylcellulose, hydroxymethylcellulose, hydroxyethylcellulose, hydroxypropylcellulose, hydroxypropylmethylcellulose and inorganic thickeners, e.g. aluminum silicates, such as, for example, organically modified or also unmodified hectorites, bentonites, or the like, or a mixture of polyethylene glycol or polyethylene glycol stearate or distearate. The thickener is present in the gel, for example, in an amount between 0.1 and 30% by weight, preferably between 0.5 and 15% by weight.

In addition, it may be advantageous to add interface-active or surface-active agents to preparations according to the invention and, for example, cationic emulsifiers, such as, in particular, quaternary surfactants.

Quaternary surfactants contain at least one N atom which is covalently bonded to 4 alkyl or aryl groups. Irrespective of the pH, this leads to a positive charge. Alkylbetaine, alkylamidopropylbetaine and alkylamidopropylhydroxysultaine are advantageous. The cationic surfactants used according to the invention can also preferably be chosen from the group of quaternary ammonium compounds, in particular benzyltrialkylammonium chlorides or bromides, such as, for example, benzyldimethylstearylammonium chloride, and also alkyltrialkylammonium salts, for example cetyltrimethylammonium chloride or bromide, alkyldimethylhydroxyethylammonium chlorides or bromides, dialkyldimethylammonium chlorides or bromides, alkylamidoethyltrimethylammonium ether sulfates, alkylpyridinium salts, for example lauryl- or cetylpyridinium chloride, imidazoline derivatives and compounds having cationic character, such as amine oxides, for example alkyldimethylamine oxides or alkylaminoethyldimethylamine oxides. In particular, cetyltrimethylammonium salts are to be used advantageously.

It is also advantageous to use cationic polymers (e.g. Jaguar C 162 [Hydroxypropyl Guar Hydroxypropyltrimonium Chloride] or modified magnesium aluminum silicates (e.g. quaternium-18 hectorite, which is available, for example, under the tradename Bentone® 38 from Rheox, or stearalkonium hectorite, which is available, for example, under the tradename Softisan® gel from Huls AG).

Preparations according to the invention can advantageously also comprise oil thickeners in order to improve the tactile properties of the emulsion and the stick consistency. Advantageous oil thickeners for the purposes of the present invention are, for example, further solids, such as, for example, hydrophobic silicone oxides of the Aerosil® type, which are available from Degussa AG. Advantageous Aerosil® products are, for example, Aerosil® OX50, Aerosil® 130, Aerosil® 150, Aerosil® 200, Aerosil® 300, Aerosil® 380, Aerosil® MOX 80, Aerosil® MOX 170, Aerosil® COK 84, Aerosil® R 202, Aerosil® R 805, Aerosil® R 812, Aerosil® R 972, Aerosil® R 974 and/or Aerosil® R 976.

In addition, metal soaps (i.e. the salts of higher fatty acids with the exception of the alkali metal salts) are advantageous oil thickeners for the purposes of the present invention, such as, for example, aluminum stearate, zinc stearate and/or magnesium stearate.

It is likewise advantageous to add amphoteric or zwitterionic surfactants (e.g. cocoamidopropylbetaine) and moisturizers (e.g. betaine) to preparations according to the invention. Amphoteric surfactants to be used advantageously are, for example, acyl/dialkylethylenediamine, for example sodium acylamphoacetate, disodium acylamphodipropionate, disodium alkylamphodiacetate, sodium acylamphohydroxypropylsulfonate, disodium acylamphodiacetate and sodium acylamphopropionate, N-alkylamino acids, for example aminopropylalkylglutamide, alkylaminopropionic acid, sodium alkylimidodipropionate and lauroamphocarboxyglycinate.

The amount of the surface-active or interface-active substances (one or more compounds) in the preparations according to the invention is preferably 0.001 to 30% by weight, particularly preferably 0.05-20% by weight, in particular 1-10% by weight, based on the total weight of the preparation.

Preparations according to the invention may also comprise active ingredients (one or more compounds) which are chosen from the group: acetylsalicylic acid, atropine, azulene, hydrocortisone and derivatives thereof, e.g. hydrocortisone-17 valerate, vitamins, e.g. ascorbic acid and derivatives thereof, vitamins of the B and D series, very favorably vitamin B1, vitamin B₁₂ and vitamin D₁, but also bisabolol, unsaturated fatty acids, namely the essential fatty acids (often also called vitamin F), in particular γ-linolenic acid, oleic acid, eicosapentaenoic acid, docosahexaenoic acid and derivatives thereof, chloramphenicol, caffeine, prostaglandins, thymol, camphor, extracts or other products of a vegetable or animal origin, e.g. evening primrose oil, borage oil or currant seed oil, fish oils, cod-liver oil and also ceramides and ceramide-like compounds, etc. It is also advantageous to choose the active ingredients from the group of refatting substances, for example purcellin oil, Eucerit® and Neocerit®.

The amount of such active ingredients (one or more compounds) in the preparations according to the invention is preferably from 0.001 to 30% by weight, particularly preferably 0.05-20% by weight, in particular 1-10% by weight, based on the total weight of the preparation.

The examples below serve to illustrate the present invention.

Preparation example Chitosan solution:

| | % by weight |
|---|---|
| Chitosan | 2.0 |
| Lactic acid (90% strength aqueous solution) | 1.2 |
| Water | ad 100.00 |
| pH about | 4.5 |

The chitosan is incorporated by adjusting a suspension of micronized chitosan in water to a pH of 4.5 using lactic acid, giving a solution of the chitosan.

Formulation example 1

| | % by weight |
|---|---|
| Glycerol | 2.00 |
| Ethanol | 70.00 |
| Bisabolol | 0.10 |
| Panthenol | 1.50 |
| Chitosan solution according to Preparation example, | 0.50 |
| corresponding to a total content of | |
| Chitosan | |
| Lactic acid | 0.33 |
| Niacinamide | 0.10 |
| Perfume | q.s. |
| Water | ad 100.00 |

The constituents (including the chitosan solution according to Preparation example) are combined at room temperature and stirred until a clear solution has formed.

Formulation example 2

| | % by weight |
|---|---|
| Glycerol | 2.00 |
| Ethanol | 70.00 |
| Bisabolol | 0.10 |
| Panthenol | 1.50 |
| Chitosan solution according to Preparation example, | 0.50 |
| corresponding to a total content of | |
| Chitosan | |
| Lactic acid | 0.33 |
| Niacinamide | 0.10 |
| Na₃HEDTA | 0.20 |
| Perfume | q.s. |
| Water | ad 100.00 |

Formulation example 3

| | % by weight |
|---|---|
| Glycerol | 2.00 |
| Ethanol | 70.00 |
| Bisabolol | 0.10 |
| Panthenol | 1.50 |
| Chitosan solution according to Preparation example, | 0.50 |
| corresponding to a total content of | |
| Chitosan | |
| Lactic acid | 0.33 |
| Niacinamide | 0.10 |
| Na₃HEDTA | 0.20 |
| Perfume | q.s. |
| Water | ad 100.00 |

Formulation example 4

| | % by weight |
|---|---|
| Glycerol | 2.00 |
| Ethanol | 70.00 |
| Bisabolol | 0.10 |
| Panthenol | 1.50 |
| Chitosan solution according to Preparation example, | 0.50 |
| corresponding to a total content of | |
| Chitosan | |
| Lactic acid | 0.33 |
| Niacinamide | 0.10 |
| Na₃HEDTA | 0.20 |
| Thickener | 0.10 |
| Perfume | q.s. |
| Water | ad 100.00 |

1. A cosmetic face tonic comprising
a) chitosan having an average molecular weight of from 10 000 to 1 000 000 g/mol and a degree of deacylation of from 10 to 99%
b) 10-80% by weight of ethanol
c) 0-5% by weight of one or more lipids,
d) 0-5% by weight of one or more fatty alcohols.

2. The use of chitosan having an average molecular weight of from 10 000 to 1 000 000 g/mol and a degree of deacylation of from 10 to 99% for the preparation of skin-friendly and/or nonsticky alcoholic cosmetic face tonics.

3. The face tonic as claimed in claim 1 or the use as claimed in claim 2, wherein the total amount of one or more chitosans is chosen from the range 0.01 to 10% by weight, advantageously 0.1 to 5% by weight, very particularly preferably 0.25 to 2.5% by weight, based on the total weight of the preparations.

4. A face tonic as claimed in claim 1 or the use as claimed in claim 2, wherein the total amount of one or more fatty alcohols in the finished cosmetic or dermatological preparations is chosen from the range 0.1-5.0% by weight, preferably 0.5-1.0% by weight, based on the total weight of the preparations.

5. The face tonic as claimed in claim 1 or the use as claimed in claim 2, wherein the total amount of lipid component or lipid components, for example advantageously one or more phospholipids, particularly advantageously one or more lecithins, in the preparations is chosen from the range 0.001 to 5% by weight, particularly preferably 0.05-2.5% by weight, in particular 0.5-1% by weight, in each case based on the total weight of the preparation.
